(19) United States
(12) Patent Application Publication
(10) Pub. No.: US 2010/0128849 A1
(43) Pub. Date: May 27, 2010

(54) DENTAL RADIOGRAPHIC FILM HOLDER

(76) Inventor: Rebecca Brewington, Springfield, OH (US)

Correspondence Address: LAW OFFICE OF JERRY D. HAYNES , P.A, P.O. Box 350392, Fort Lauderdale, FL, 33335, US

(21) Appl. No.: 12/416,525

(22) Filed: Apr. 1, 2009

Related U.S. Application Data
(60) Provisional application No. 61/117,986, filed on Nov. 26, 2008.

(51) Int. Cl. A61B 6/14

(52) U.S. Cl. 378/168

(57) ABSTRACT

A dental radiographic film holder includes a support member, a mouthpiece member, and a flexible material. The support member is adapted to hold a dental radiographic film. The mouthpiece member extends perpendicularly from a portion of the support member. The mouthpiece member is adapted to be secured between at least one pair of teeth for aligning the support member with the at least one pair of teeth. The flexible material is disposed on the mouthpiece member. The flexible material is adapted to conform to contours of the at least one pair of teeth securing the mouthpiece member therebetween.

### CROSS-REFERENCE TO RELATED APPLICATIONS

The present invention claims priority under 35 United States Code, Section 119 on the U.S. Provisional Patent Application No. 61/117,986 filed on Nov. 26, 2008, the disclosure of which is incorporated by reference.

### FIELD OF THE INVENTION

The present invention relates generally, to dental radiology, and, more particularly, to a dental radiographic film holder for aiding in generating dental radiographs.

### BACKGROUND OF THE INVENTION

People make periodic visits to a dentist to maintan a healthy condition of their teeth. During such visits, people may undergo various dental examination procedures, such as a dental radiographic examination in which images of teeth are taken for diagnosing and monitoring disorders related thereto. Typically, the dental radiographic examination is performed by utilizing a dental radiographic film. The dental radiographic examination may be broadly divided into two categories: an intraoral x-ray examination, in which the dental radiographic film is positioned within an oral cavity of a patient undergoing such examination; and an extraoral x-ray examination in which the dental radiographic film is positioned outside the oral cavity of the patient. Moreover, both the intraoral x-ray examination and the extraoral x-ray examination utilize various techniques for performing the dental radiographic examinations effectively.

For example, the intraoral x-ray examination may utilize a bitewing x-ray technique. The bitewing x-ray technique enables in detecting many dental disorders, such as tartar or calculus on roots, cavities between teeth, periodontal conditions and bone levels between teeth, and abscesses. Typically, the bitewing x-ray technique is performed with the help of a dental radiographic film holder. The dental radiographic film holder is capable of securing the dental radiographic film thereon. Further, the dental radiographic film holder includes a mouthpiece member (bite tab) adapted to be secured between teeth of the patient for positioning the dental radiographic film in a proper position. Specifically, the mouthpiece member is positioned within the oral cavity and is secured between mandibular and maxillary teeth of the patient in a manner such that the dental radiographic film is positioned behind the teeth for which a dental radiograph is to be generated. Accordingly, upon exposing the teeth to x-rays, the dental radiograph is generated on the dental radiographic film.

Presently, many dental radiographic film holders are known in the art thar may be used for performing dental radiographic examinations. Usually, such dental radiographic film holders are composed of a hard material or a moderately flexible material. Specifically, a patient may find it uncomfortable to hold the dental radiographic film holder for a prolonged duration of time due to the hard or inflexible nature of the mouthpieces of the dental radiographic film holder. Accordingly, the dental radiographic film holder may be displaced from the proper position thereof, which may cause unclear dental radiographs of the teeth. Additionally, such dental radiographic film holders usually possess rough and sharp edges which may contact oral parts, such as gums and lips of a patient, thereby causing discomfort and inconvenience to the patient. Moreover, upon securing the mouthpieces between the teeth, the patient may experience an unpleasant smell and an unpleasant taste of the mouthpieces, which may stimulate a gag or choke response in the patient.

Accordingly, there exists a need for a dental radiographic film holder that avoids discomfort to a patient holding the dental radiographic film holder while performing a dental radiographic examination. Also, there exists a need for a dental radiographic film holder that precludes an experience of an unpleasant taste and an unpleasant smell during a dental radiographic examination.

### SUMMARY OF THE INVENTION

In view of the forgoing disadvantages inherent in the prior art, the general purpose of the present invention is to provide a dental radiographic film holder, to include all advantages of the prior art, and to overcome the drawbacks inherent in the prior art.

An object of the present invention is to provide a dental radiographic film holder that avoids discomfort to a patient holding the dental radiographic film holder while performing a dental radiographic examination.

Another object of the present invention is to provide a dental radiographic film holder that precludes an experience of an unpleasant taste and an unpleasant smell during a dental radiographic examination.

To achieve the above objects, in one aspect of the present invention, a dental radiographic film holder is provided. The dental radiographic film holder comprises a support member, a mouthpiece member, and a flexible material. The support member is adapted to hold a dental radiographic film. The mouthpiece member is extending perpendicularly from a portion of the support member. The mouthpiece member is adapted to be secured between at least one pair of teeth for aligning the support member with the at least one pair of teeth. The flexible material is disposed on the mouthpiece member. The flexible material is adapted to conform to contours of the at least one pair of teeth securing the mouthpiece member therebetween.

In another aspect of the present invention, the flexible material of the dental radiographic film holder comprises a flavor which is adapted to be released upon the mouthpiece member being secured between the at least one pair of teeth.

These together with the other aspects of the present invention, along with the various features of novelty that characterize the present invention, is pointed out with particularity in the claims annexed hereto and forms a part of the present invention. For a better understanding of the present invention, its operating advantages, and the specified object attained by its uses, reference should be made to the accompanying drawings and descriptive matter in which there are illustrated exemplary embodiments of the present invention.

### BRIEF DESCRIPTION OF THE DRAWING

The advantages and features of the present invention will become better understood with reference to the following detailed description and claims taken in conjunction with the accompanying drawings, in which:

FIG. 1 illustrates a perspective view of a dental radiographic film holder, in accordance with an exemplary embodiment of the present invention; and

FIG. 2 illustrates an environment in which the dental radiographic film holder of FIG. 1 is utilized, in accordance with an exemplary embodiment of the present invention.

Like reference numerals refer to like parts throughout the description of several views of the drawings.

### DETAILED DESCRIPTION OF THE INVENTION

The exemplary embodiments described herein detail for illustrative purposes are subject to many variations in structure and design. It should be emphasized, however, that the present invention is not limited to a particular dental radiographic film holder, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The terms “a” and “an,” as used herein do not denote a limitation of quantity, but rather denote the presence of at least one of the referenced item.

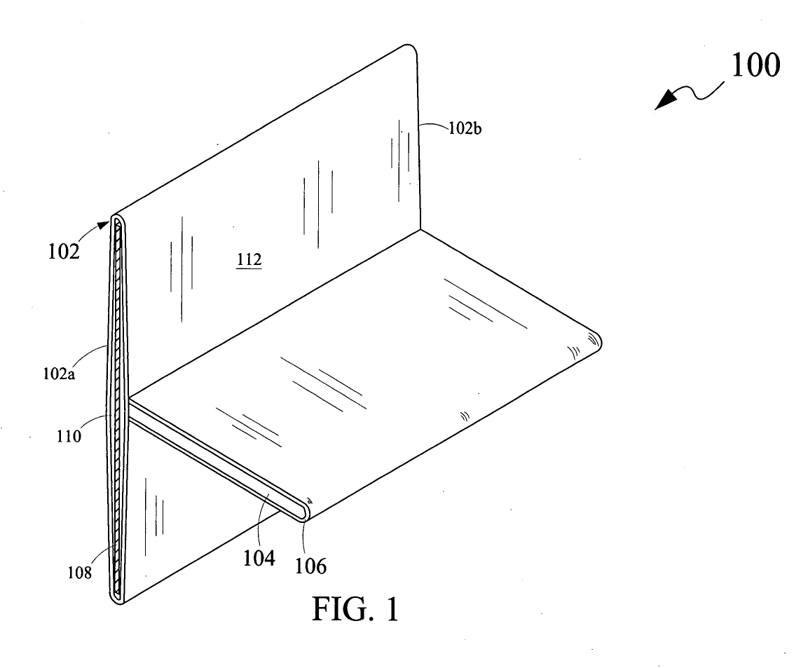

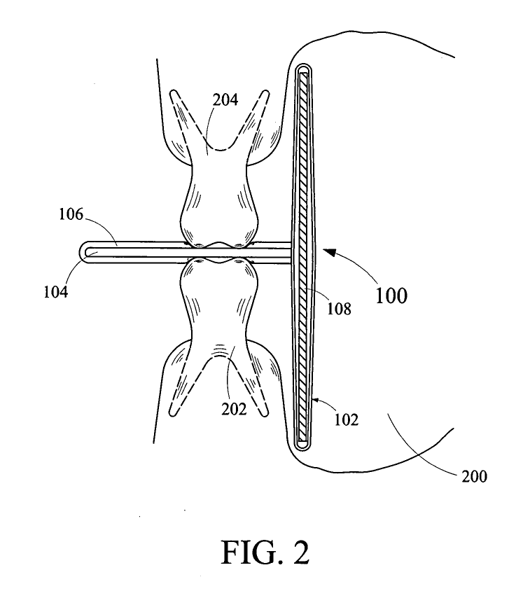

The present invention provides a dental radiographic film holder capable of holding a dental radiographic film. Specifically, the dental radiographic film holder of the present invention is capable of providing better securement of the dental radiographic film within an oral cavity of a patient undergoing a dental radiographic examination, such as a bitewing x-ray technique. More specifically, a mouthpiece of the dental radiographic film holders is made of a flexible material that aligns with contours of the teeth of the patient thereby preventing improper securement of the dental radiographic film between the teeth. Further, the dental radiographic film holder is adapted to avoid discomfort to the patient while performing a dental radiographic examination. Furthermore, the dental radiographic film holder of the present invention is capable of precluding an experience of an unpleasant taste and an unpleasant smell which may be experienced by the patient during the dental radiographic examination. The dental radiographic film holder of the present invention is explained in conjunction with FIGS. 1 and 2.

Referring to FIG. 1, a perspective view of a dental radiographic film holder 100 (hereinafter referred to as a holder 100) is illustrated, in accordance with an exemplary embodiment of the present invention. The holder 100 includes a support member 102, a mouthpiece member 104 extending perpendicularly from the support member 102, and a flexible material 106 disposed on the mouthpiece member 104.

The support member 102 of the holder 100 is adapted to hold a dental radiographic film 108 (hereinafter referred to as a film 108). Specifically, in one embodiment of the present invention, the support member 102 includes a longitudinal cavity 110 capable of removably receiving the film 108 therein. As shown in FIG. 1, the longitudinal cavity 110 is configured to extend between end faces 102a and 102b of the support member 102. It will be obvious to those skilled in the art that the support member 102 of the holder 100 may include any suitable fastening mechanism configured on a front surface (not shown) of the support member 102, which is capable of holding the film 108 on the front surface.

In the present embodiment, the support member 102 of the holder 100 is configured to assume a rectangular shape. However, it will be obvious to those skilled in the art that the support member 102 may be configured to assume other shapes apart from the rectangular shape, such as a square shape and an elliptical shape. Further, the support member 102 may be configured to assume various sizes depending on a size of dental radiographic film, such as the film 108. The support member 102 is adapted to be received within the oral cavity of a patient undergoing a dental radiographic examination. Accordingly, in the present embodiment, corners of the support member 102 are rounded for providing smooth contact surfaces to the oral parts, such as gums and lips, of the patient. Specifically, when the holder 100 is received within the oral cavity of the patient, the rounded corners of the support member 102 avoid causing irritation and discomfort to oral parts of the patient.

As explained herein, the mouthpiece member 104 extends from the support member 102. Specifically, in the present embodiment, the mouthpiece member 104 is coupled to the support member 102. However, it will be evident to those skilled in the art that the mouthpiece member 104 may be configured to form an integral part of the support member 102. Further, in the present embodiment, the mouthpiece member 104 extends perpendicularly from a portion (not numbered) of the support member 102. More specifically, as shown in FIG. 1, the mouthpiece member 104 extends perpendicularly from an intermediate portion of a back surface 112 of the support member 102. It will be obvious to a person skilled in the art that the mouthpiece member 104 may extend from any other convenient portion (apart from the intermediate portion) of the back surface 112. Furthermore, in the present embodiment, the mouthpiece member 104 is also configured to assume a rectangular shape and configured to have rounded corners, similar to that of the support member 102. Accordingly, the description thereof is avoided for the sake of brevity.

The mouthpiece member 104 of the present invention is adapted to be secured between at least one pair of teeth when the support member 102 is positioned within the oral cavity of the patient for aligning the support member 102 with the at least one pair of teeth. The positioning of the support member 102 in the oral cavity will be further explained in detail in conjunction with FIG. 2. As explained herein, the flexible material 106 is disposed on the mouthpiece member 104. Accordingly, the flexible material 106 is adapted to conform to contours of the at least one pair of teeth when the mouthpiece member 104 is secured therebetween. Specifically, in one embodiment of the present invention, the flexible material 106 is a consumable latex product, such as bubble gum. Further, in one embodiment of the present invention, the flexible material 106 includes a flavor. For example, the flavor of the flexible material 106 may include at least one of a cinnamon flavor, a lemon flavor, a mint flavor, a cherry flavor, a strawberry flavor, and a combination thereof. The above mentioned flavors are to be considered as being examples only and not as limiting the scope of the present invention.

FIG. 2 illustrates an environment in which the holder 100 of the present invention is utilized, in accordance with an exemplary embodiment of the present invention. Specifically, FIG. 2 illustrates, a side view of the holder 100 configured within a mouth (not shown) of a patient. More specifically, as shown in FIG. 2, the support member 102, holding the film 108 therein, is received in an oral cavity 200 of the mouth of the patient. The mouthpiece member 104 is secured between at least one pair of teeth, such as a mandibular tooth 202 and a maxillary tooth 204. For the description of the present invention, it is assumed that the at least one pair of teeth, such as the mandibular tooth 202 and the maxillary tooth 204, require to be examined and accordingly a radiograph of the at least one pair of teeth is to be generated. It will be evident to a person skilled in the art that the mouthpiece member 104 may be secured between a suitable number of pair of mandibular teeth and maxillary teeth of the patient depending upon a width of the mouthpiece member 104.

Upon securing the mouthpiece member 104 between the least one pair of teeth, the flexible material 106 conforms to the contours of the at least one pair of teeth. Specifically, the flexible material 106 is capable of conforming to the contours of the at least one pair of teeth, such as the mandibular tooth 202 and the maxillary tooth 204. Accordingly, the holder 100 is secured in a better manner within the oral cavity 200 without causing any discomfort to the patient over a prolonged duration of time. Further, upon securement of the mouthpiece member 104 between the least one pair of teeth, the support member 102 is aligned with the at least one pair of teeth, as shown in FIG. 2. Specifically, the film 108 received within the support member 102 is aligned with the mandibular tooth 202 and the maxillary tooth 204. More specifically, the film 108 is positioned within the oral cavity 200 and behind the mandibular tooth 202 and the maxillary tooth 204 such that when the mandibular tooth 202 and the maxillary tooth 204 are exposed to x-rays, a radiograph thereof is generated on the film 108.

Further, as explained herein the flexible material 106 includes the flavor. Accordingly, the flavor is adapted to be released when the mouthpiece member 104 is secured between the at least one pair of teeth, such as the mandibular tooth 202 and the maxillary tooth 204. Therefore, the released flavor from the flexible material 106 enables in avoiding an experience of an unpleasant smell and an unpleasant taste which may be experienced by the patient upon securing the mouthpiece member 104 between the at least one pair of teeth. Further, the flavor released from the flexible material 106 enables in considerably reducing a feeling of choking or gagging in the patient.

As explained herein, the dental radiographic film holder, such as the holder 100, enables in providing better securement of a dental radiographic film while performing a dental radiographic examination, such as a bitewing x-rays technique. The better securement prevents a displacement of the dental radiographic film holder from a proper position thereof, thereby enabling clear radiographs of teeth of the patient. Furthermore the dental radiographic film holder includes a flavor material which enables in providing a pleasant dental radiography experience to the patients, particularly for children. Moreover, the dental radiographic film holder includes rounded corners which improves patient comfort and reduces a risk of injury of oral parts, such as gums and lips. Additionally, the dental radiographic film holder may be configured to assume various sizes depending on age of a patient and/ or size of teeth of the patient. Accordingly, the dental radiographic film holder may be designed for individuals of different age groups. Further, the dental radiographic film holder may be sold in a packet including about 200 to 500 units of the dental radiographic film holders.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, to thereby enable others skilled in the art to best utilize the present invention and various embodiments with various modifications as are suited to the particular use contemplated. It is understood that various omission and substitutions of equivalents are contemplated as circumstance may suggest or render expedient, but such are intended to cover the application or implementation without departing from the spirit or scope of the claims of the present invention.

What is claimed is:

1. A dental radiographic film holder comprising:
a support member adapted to hold a dental radiographic film;
a mouthpiece member extending perpendicularly from a portion of the support member, the mouthpiece member adapted to be secured between at least one pair of teeth for aligning the support member with the at least one pair of teeth; and
a flexible material disposed on the mouthpiece member, the flexible material adapted to conform to contours of the at least one pair of teeth securing the mouthpiece member therebetween.

2. The dental radiographic film holder of claim 1, wherein the support member includes a cavity capable of removably receiving the dental radiographic film.

3. The dental radiographic film holder of claim 1, wherein the flexible material is a consumable latex product.

4. The dental radiographic film holder of claim 1, wherein the flexible material comprises a flavor adapted to be released upon the mouthpiece member being secured between the at least one pair of teeth.

5. The dental radiographic film holder of claim 4, wherein the flavor comprises at least one of a cinnamon flavor, a lemon flavor, a mint flavor, a cherry flavor, a strawberry flavor, and a combination thereof.

6. The dental radiographic film holder of claim 1, wherein the at least one pair of teeth comprises a mandibular tooth and a maxillary tooth.
